(19) United States
(12) Patent Application Publication
(10) Pub. No.: US 2026/0109664 A1
(43) Pub. Date: Apr. 23, 2026

(54) COMPOUNDS FOR USE AS MEDICAMENTS

(71) Applicant: Chariot Innovations LTD, London (GB)

(72) Inventors: Lisa Dawson, London (GB); Mark Harrison, London (GB)

(21) Appl. No.: 19/118,734

(22) PCT Filed: Oct. 12, 2023
(86) PCT No.: PCT/GB2023/052645

§ 371 Date: Apr. 4, 2025

(30) Foreign Application Priority Data
Oct. 12, 2022 (GB) 2215058.5

(51) Int. Cl. C07C 233/11; A61K 31/165; A61K 31/192; A61K 31/216; A61K 31/277; C07C 63/04; C07C 255/36

(52) U.S. Cl. CPC C07C 233/11; A61K 31/165; A61K 31/192; A61K 31/216; A61K 31/277; C07C 63/04; C07C 255/36

(57) ABSTRACT

The application relates to compound of Formula (I) or a pharmaceutically acceptable salt, solvate or prodrug thereof, for use as a medicament.

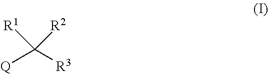

### FIELD OF THE INVENTION

Described herein are compounds for use as medicaments, as well as compositions comprising said compounds.

### BACKGROUND OF THE INVENTION

Bacterial infections are prevalent around the world. Of particular concern are infections that are picked up by patients whilst in a healthcare environment. These are typically known as nosocomial infections, or healthcare-associated infections. Many nosocomial infections are becoming more difficult to treat due to increasing resistance by the bacteria to conventional antibiotics.

For example, Clostridioides difficile (C. difficile) is a nosocomial pathogen that causes significant mortality and morbidity globally. C. difficile primarily affects patients who have been treated with broad spectrum antibiotics for an unrelated condition, resulting in damage to the gut microbiome and a resultant loss of colonisation resistance to C. difficile, with patients who receive longer courses of therapy being at greater risk than those receiving short courses.

C. difficile produces 4-methylphenol (p-cresol) by the fermentation of 4-hydroxyphenylalanine (p-tyrosine) via the intermediate 4-hydroxyphenylacetic acid (p-hydroxyphenylacetic acid (p-HPA)) or by the conversion of exogenous 4-hydroxyphenylacetic acid. Both pathways utilise the action of HpdBCA decarboxylase, which is encoded by the hpdBCA operon. 4-methylphenol selectively inhibits growth of Gram-negative bacteria of the Gammaproteobacteria class, including Escherichia coli, Proteus mirabillis and Klebsiella oxytoca. The production of 4-methylphenol by C. difficile in the human gut may, therefore, reduce gut microbial diversity and allow for the proliferation of C. difficile, which can result in sustained or recurrent C. difficile infections. Less than 1% of the currently sequenced bacteria in the gut microbiome encode the decarboxylase required to make 4-methylphenol from p-HPA. Other bacteria are known to produce 4-methylphenol.

Whilst treatment of C. difficile is effective with metronidazole, vancomycin or fidaxomicin, a major feature of C. difficile infection (CDI) is the high proportion of patients (20-30%) who suffer from recurrence (often multiple recurrences) either as a result of reinfection or relapse. Furthermore, existing antibiotics may have a negative impact on the healthy protective microbiome, which is important in preventing diseases such as cancer and diabetes, as well as conditions that may be linked to the health of the microbiome, such as autism spectrum disorders, for example.

Therefore, treatments that prevent the recurrence of C. difficile infections and do not have significant effects on the protective microbiome are in high demand.

### SUMMARY OF THE INVENTION

According to a first aspect of the invention, there is provided a compound of Formula (I):

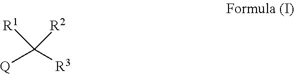

or a pharmaceutically acceptable salt, solvate or prodrug thereof, for use as a medicament, wherein:
Q is a substituted or unsubstituted cyclic group;
each of R¹ and R² together represent ═O or is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α}C(O)OR^{α}, —R^{α}C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}; and
R³ is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α}C(O)OR^{α}, —R^{α}C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}, wherein each R^{α} is independently selected from a methylene, C₁-C₂₀ alkyl, C₂-C₂₀ alkenyl, C₂-C₂₀ alkynyl or C₃-C₂₀ cyclic group, wherein any —R^{α} may optionally be substituted with one or more methylene, C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups and wherein each n is independently an integer from 1 to 20.

In some embodiments, Q is a substituted or unsubstituted 3- to 10-membered cyclic group.

In some embodiments, Q is a substituted or unsubstituted 5-membered or 6-membered cyclic group, optionally wherein the cyclic group is a heterocyclic group.

In some embodiments, Q is a substituted or unsubstituted aryl, cyclohexyl, pyridyl or piperidinyl group.

In some embodiments, each of R¹ and R² together represent ═O or R¹ is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α} and R² is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}, wherein each R^{α} is independently selected from a methylene, C₁-C₂₀ alkyl, C₂-C₂₀ alkenyl, C₂-C₂₀ alkynyl or C₃-C₂₀ cyclic group, wherein any —R^{α} may optionally be substituted with one or more methylene, C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups and wherein each n is independently an integer from 1 to 20.

In some embodiments, R³ is —R^{α}OH, OH, —CO₂R^{α}, —R^{α} CO₂R^{α}, —R^{α} CO₂H, —CH₃, —CN, —R^{α}CN, —CₙH₂ₙ₋₂(O)NH₂, —CₙH₂ₙ₋₂(O)NHR^{α} or —CₙH₂ₙ₋₂(O)N(R^{α})R^{α}.

In some embodiments, n is an integer from 1 to 5.

In some embodiments, R³ is —OH, —COOH, —C(O)OCH₃, —C(O)OCH₂CH₃, —CH₂C(O)OCH₃, —CH₂C(O)OCH₂CH₃, —CH₂C(O)OH, —CH₂CH₂C(O)OH, —CH₂CH₂C(O)CH₃, —CH₂CH₂C(O)OCH₂CH₃, —CH₃, —CN, —CH₂CN, —C₂H₄CN, —C(O)NH₂, —C(O)NCH₃, —C(O)N(H)CH₂CH₃, —CH₂(O)NH₂, —CH₂(O)NHCH₃ or —CH₂(O)N(CH₃)CH₃.

In some embodiments, the compound is selected from the group consisting of:

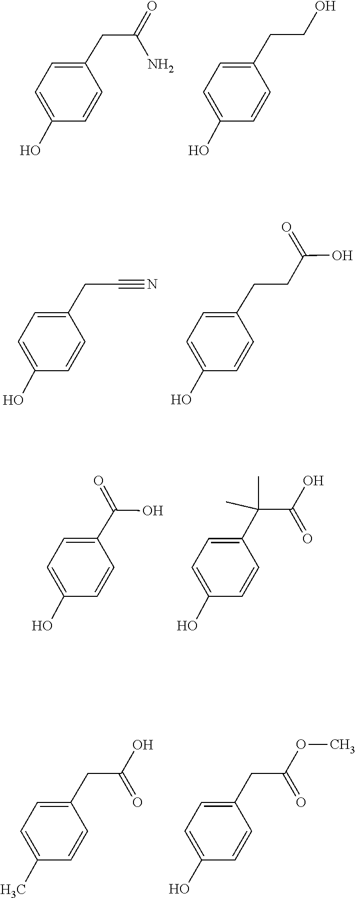

In some embodiments, the compound is for use in the treatment of a bacterial infection.

In some embodiments, the compound is for use in the inhibition of 4-methylphenol production by bacteria.

In some embodiments, the compound is for use in the treatment of Clostridioides difficile infection.

According to a second aspect of the invention, a pharmaceutically acceptable salt, solvate or prodrug of a compound of the first aspect is provided.

According to a third aspect of the invention, a pharmaceutical composition comprising a compound of the first aspect, or a pharmaceutically acceptable salt, solvate or prodrug of the second aspect, and a pharmaceutically acceptable excipient, is provided.

According to a fourth aspect of the invention, a method of inhibiting the production of 4-methylphenol by a bacteria, the method comprising the use of a compound of the first aspect, a pharmaceutically acceptable salt, solvate or prodrug of the second aspect, or a composition of the third aspect is provided.

According to a fifth aspect of the invention, a method of treatment or prevention of a disease, disorder or condition, the method comprising the step of administering an effective amount of a compound of the first aspect, a pharmaceutically acceptable salt, solvate or prodrug of the second aspect, or a composition of the third aspect is provided.

### BRIEF DESCRIPTION OF THE DRAWINGS

FIGS. 1a to 5b are growth curves for C. difficile 630Δerm and E. coli with compounds disclosed herein;

FIG. 6 shows viable cell data, determined by colony forming unit (CFU), for C. difficile, a mutant of C. difficile and its complemented strain when grown with E. coli;

FIGS. 7 to 13 show CFU data for C. difficile and E. coli when treated with the compounds described herein; and

FIGS. 14 and 15 are HPLC data.

### DETAILED DESCRIPTION OF THE INVENTION

A first aspect of the invention provides a compound of Formula (I):

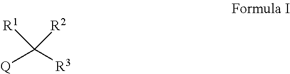

or a pharmaceutically acceptable salt, solvate or prodrug thereof, for use as a medicament, wherein:
Q is a substituted or unsubstituted cyclic group;
each of R¹ and R² together represent ═O or is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}; and
R³ is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}, wherein each R^{α} is independently selected from a C₁-C₂₀ alkyl, C₂-C₂₀ alkenyl, C₂-C₂₀ alkynyl or C₃-C₂₀ cyclic group, wherein any —R^{α} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups and wherein each n is independently an integer from 1 to 20.

It has been found that compounds according to Formula (I) may be used to inhibit the growth of bacteria. In particular, the compounds may be used to inhibit the growth of the bacterium C. difficile. Furthermore, the compounds may be suitable for treating bacterial infections, in particular C. difficile infections.

An “alkyl” substituent group or an alkyl moiety in a substituent group may be linear (i.e. straight-chained) or branched. Examples of alkyl groups/moieties include methylene (—CH₂—), methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-2-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O or S, in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O or S, in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl, thiomorpholinyl, oxetanyl, thietanyl, pyrazolidinyl, imidazolidinyl, dioxolanyl, oxathiolanyl, thianyl and dioxanyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

In some embodiments, Q is a substituted or unsubstituted 3- to 10-membered cyclic group.

In some embodiments, Q is an unsubstituted 5-membered or 6-membered cyclic group. Optionally, the cyclic group is heterocyclic and may be heteroaromatic. For example, Q may be a heteroaryl group. A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

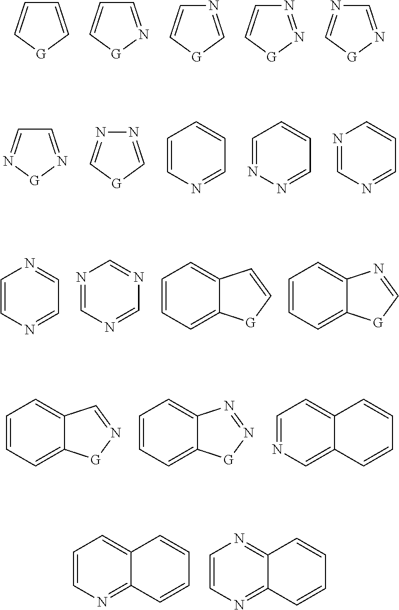

wherein G═O, S or NH.

In some embodiments, Q is:

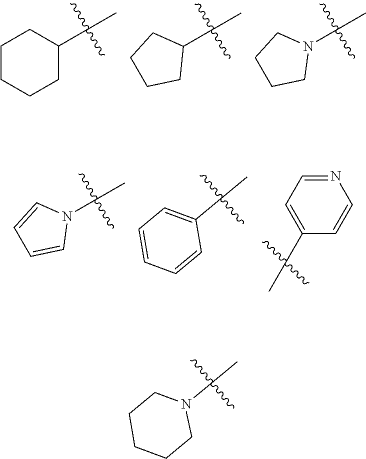

In some embodiments, Q is a substituted 5-membered or 6-membered cyclic group. Optionally, the cyclic group is a heterocyclic. In some embodiments, Q is:

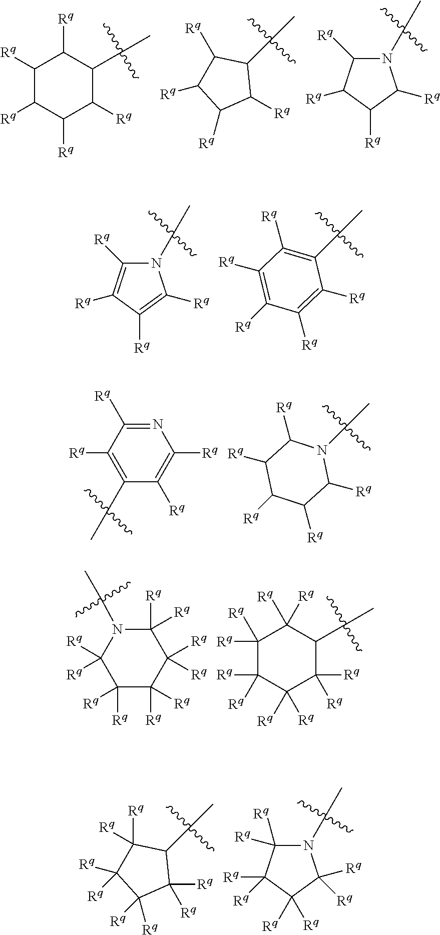

wherein each R^{q} is independently selected from —H, —R^{β}, —Cl, —Br, —I, —F, —OH, —OC(O)R^{β}, —OR^{β}, —OCH₂NHR^{β}, —OCHR^{β}NHR^{β}, —OCH₂NR^{β}R^{β}, —NO₂, —NH₂, —N₃, —SH, —SO₂H, —SO₂NH₂, —CO₂H, —CN, —C(O)NH₂, —CONHR^{β}, —C(O)NR^{β}₂, —C(O)OR^{β}, —R^{β}C(O)OR^{β}, —R^{β}C(O)OH, —CH₃, —CₙH₂ₙ₋₂(O)NH₂, —CₙH₂ₙ₋₂(O)NHR^{β}, or —CₙH₂ₙ₋₂(O)N(R^{β})R^{β}, wherein each R^{β} is independently selected from a C₁-C₂₀ alkyl, C₂-C₂₀ alkenyl, C₂-C₂₀ alkynyl or C₃-C₂₀ cyclic group, wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups and wherein each n is independently an integer from 1 to 20.

In the context of the present specification, unless otherwise stated, a Cₓ-C_{y} group is defined as a group containing from n to n carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are to be counted as carbon atoms when calculating the number of carbon atoms in a Cₓ-C_{y} group. For example, a morpholinyl group is to be considered a C₆ heterocyclic group, not a C₄ heterocyclic group.

For the purposes of the present specification, where it is stated that a first atom or group is “directly attached” to a second atom or group it is to be understood that the first atom or group is covalently bonded to the second atom or group with no intervening atom(s) or groups being present. So, for example, for the group (C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

In some embodiments, Q is:

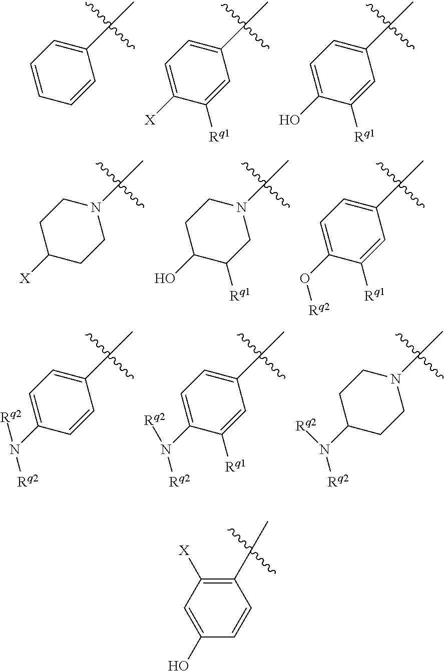

wherein X is halo,
each R^{q1} is independently selected from —H, —R^{β}, —Cl, —Br, —I, —F, —OH, —OC(O)R^{β}, —OR^{β}, —OCH₂NHR^{β}, —OCHR^{β}N₂R^{β}, —OCH₂NR^{β}R^{β}, —NO₂, —NH₂, —N₃, —SH, —SO₂H, —SO₂NH₂, —CO₂H, —CN, —C(O)NH₂, —CONHR^{β}, —C(O)NR^{β}₂, —C(O)OR^{β}, —R^{β}C(O)OR^{β}, —R^{β}C(O)OH, —CH₃, —CₙH₂ₙ₋₂(O)NH₂, —CₙH₂ₙ₋₂(O)NHR^{β}, or —CₙH₂ₙ₋₂(O)N(R^{β})R^{β}, wherein each R^{β} is independently selected from a C₁-C₂₀ alkyl, C₂-C₂₀ alkenyl, C₂-C₂₀ alkynyl or C₃-C₂₀ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups, wherein each n is independently an integer from 1 to 20; and
each R^{q2} is independently selected from H, C₁-C₂₀ alkyl, C₂-C₂₀ alkenyl, C₂-C₂₀ alkynyl or C₃-C₂₀ cyclic group.

In some embodiments, Q is:

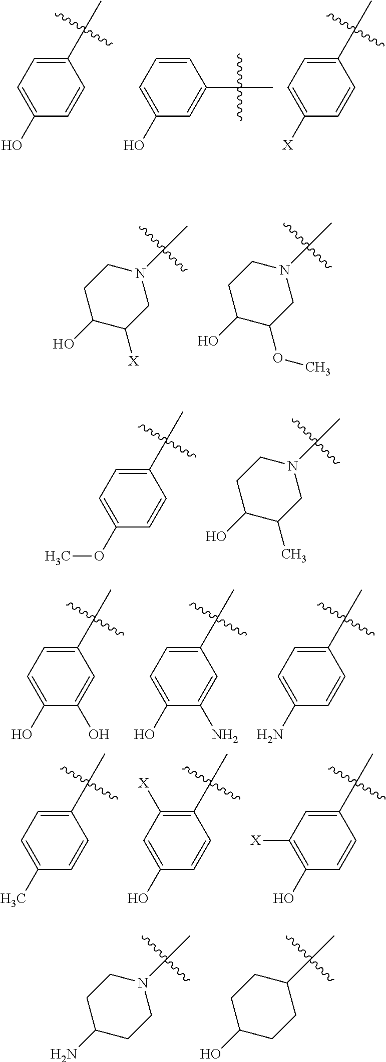

wherein each X is independently a halo or —CH₃. In preferred embodiments, each X is independently selected from the group consisting of —F, —Br and —Cl.

In preferred embodiments, Q is selected from the group consisting of:

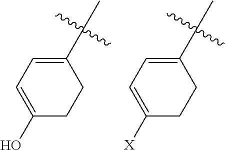

wherein X is a halo. In preferred embodiments, X is —CH₃, —Cl or —Br. In some particularly preferred embodiments, X is —Br or CH₃.

R¹ and R² can be the same or different. In some embodiments, each of R¹ and R² of Formula (I) may together represent ═O. In some embodiments, at least one of R¹ and R² is selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}, wherein each R^{α} is independently selected from a methylene, C₁-C₂₀ alkyl, C₂-C₂₀ alkenyl, C₂-C₂₀ alkynyl or C₃-C₂₀ cyclic group, wherein any —R^{α} may optionally be substituted with one or more methylene, C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups and wherein each n is independently an integer from 1 to 20.

In some embodiments, at least one of R¹ and R² is —H, both R¹ and R² are —H, R¹ is —H and R² is —C₁ to —C10 alkyl, both R¹ and R² are independently —C₁ to —C₁₀ alkyl, R¹ is H and R² is OH or R¹ is H and R² is NH₂. In some embodiments, R¹ is —H and R² is —C₁ to —C₃ alkyl or both R¹ and R² are independently —C₁ to —C₃ alkyl. In some embodiments, R¹ is —H and R² is methyl or ethyl or both R¹ and R² are independently methyl or ethyl.

R³ is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}, wherein each R^{α} is independently selected from a C₁-C₂₀ alkyl, C₂-C₂₀ alkenyl, C₂-C₂₀ alkynyl or C₃-C₂₀ cyclic group, wherein any —R^{α} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups, and wherein each n is independently an integer from 1 to 20.

In some embodiments, R³ comprises a carboxylic acid group, an ester group, an alcohol group, a nitrile group, an amine group or an ether group.

In some embodiments where R³ comprises a carboxylic acid group, R³ can be —R^{α}CO₂H, wherein R^{α} is selected from a methylene, C₁-C₂₀ alkyl, C₂-C₂₀ alkenyl, C₂-C₂₀ alkynyl or C₃-C₂₀ cyclic group, wherein —R^{α} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

In some embodiments where R³ comprises an ester group, R³ can be —R^{α}C(O)OR^{α}, wherein R^{α} is selected from a methylene, C₁-C₂₀ alkyl, C₂-C₂₀ alkenyl, C₂-C₂₀ alkynyl or C₃-C₂₀ cyclic group, wherein —R^{α} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

In some embodiments where R³ comprises an alcohol group, R³ can be —R^{α}OH, wherein R^{α} is selected from a methylene, C₁-C₂₀ alkyl, C₂-C₂₀ alkenyl, C₂-C₂₀ alkynyl or C₃-C₂₀ cyclic group, wherein —R^{α} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

In some embodiments, where R³ comprises a nitrile group, R³ can be —R^{α}CN, wherein R^{α} is selected from a methylene, C₁-C₂₀ alkyl, C₂-C₂₀ alkenyl, C₂-C₂₀ alkynyl or C₃-C₂₀ cyclic group, wherein —R^{α} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

In some embodiments, the compound has the following formula:

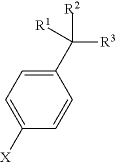

wherein each of R¹ and R² together represent ═O or is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α};
R³ is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}; and
X is an —OH or a halo, wherein each R^{α} is independently selected from a methylene, C₁-C₂₀ alkyl, C₂-C₂₀ alkenyl, C₂-C₂₀ alkynyl or C₃-C₂₀ cyclic group, wherein any —R^{α} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups, and wherein each n is independently an integer from 1 to 20. Preferably, X is —OH, —Cl or —Br. In particularly preferred embodiments, X is —OH or —Br.

In some embodiments, the compound has the following formula:

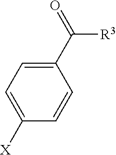

wherein R³ is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}; and
X is an —OH or a halo, wherein each R^{α} is independently selected from a methylene, C₁-C₂₀ alkyl, C₂-C₂₀ alkenyl, C₂-C₂₀ alkynyl or C₃-C₂₀ cyclic group, wherein any —R^{α} may optionally be substituted with one or more methylene, C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups, and wherein each n is independently an integer from 1 to 20. Preferably, X is —OH, —Cl or —Br. In particularly preferred embodiments, X is —OH or —Br.

In some embodiments, the compound has the following formula:

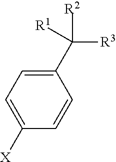

wherein each of R¹ and R² is independently selected from —H, wherein R³ is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}; and
X is an —OH or a halo, wherein each R^{α} is independently selected from a C₁-C₂₀ alkyl, C₂-C₂₀ alkenyl, C₂-C₂₀ alkynyl or C₃-C₂₀ cyclic group, wherein any —R^{α} may optionally be substituted with one or more methylene, C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups and wherein each n is independently an integer from 1 to 20. Preferably, X is —OH, —Cl or —Br. In particularly preferred embodiments, X is —OH or —Br.

In some embodiments, the compound has the following formula:

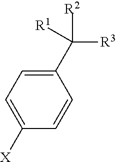

wherein each of R¹ and R² is —H, wherein R³ is independently selected from —CO₂H, —CₙH₂ₙCO₂H, —C(O)NH₂, —C(O)OR^{α}, —CₙH₂ₙC(O)R^{α}, or —CN, wherein each R^{α} is independently selected from a C₁-C₄ alkyl, each n is independently an integer from 1 to 4, and wherein X is a —OH, alkyl, or a halo. Preferably, X is —OH, —CH₃, —Cl or —Br. In particularly preferred embodiments, X is —OH or —Br

In some embodiments, the compound has the following formula:

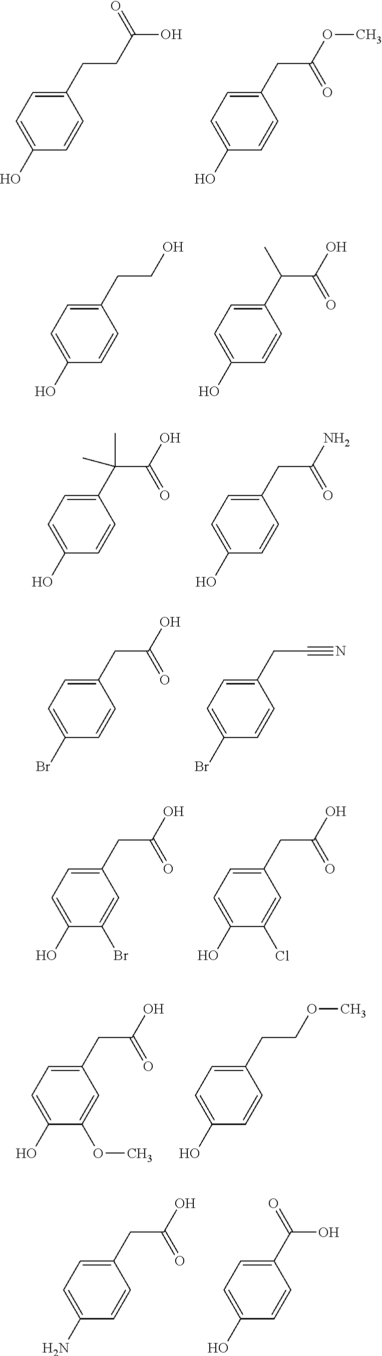

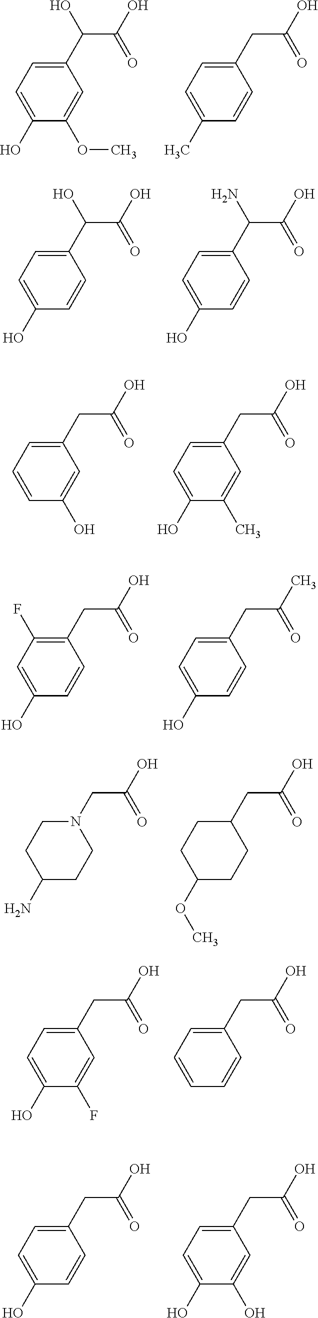

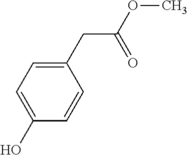

In preferred embodiments, the compound has the following formula:

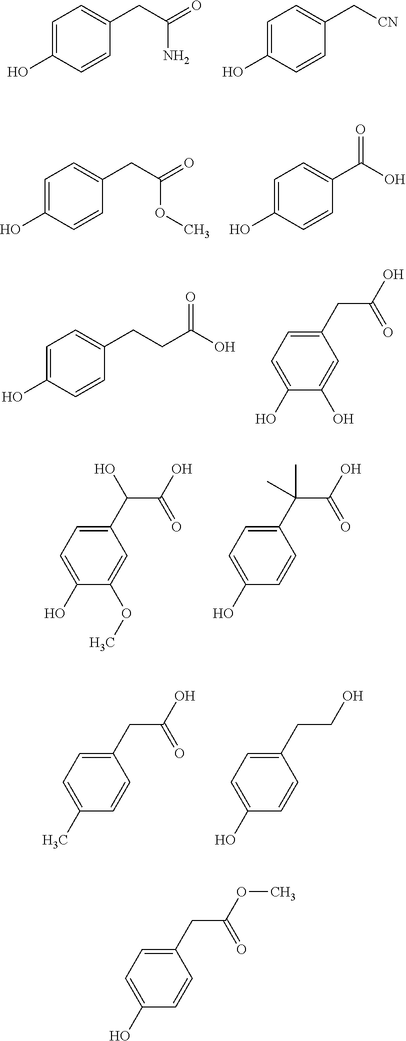

The compounds described herein have been found to reduce the proliferation of C. difficile when in the presence of other bacteria, such as E. coli. Without wishing to be bound by theory, it is believed that compounds described herein inhibit the production of 4-methylphenol by C. difficile, which is toxic to bacteria, such as E. coli. By inhibiting the production of 4-methylphenol, E. coli can effectively compete against C. difficile and gut microbial diversity may be preserved.

A second aspect of the invention provides a pharmaceutically acceptable salt, solvate or prodrug of any compound of the first aspect of the invention.

The compounds of the present invention can be used both in their free base form and their acid addition salt form. For the purposes of this invention, a “salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid-addition salts.

The compounds of the present invention can also be used both, in their free acid form and their salt form. For the purposes of this invention, a “salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts of the present invention may be anhydrous or in the form of a hydrate (e.g. a hemihydrate, monohydrate, dihydrate or trihydrate) or other solvate. Such solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, salts, solvates and prodrugs of the present invention may be in any polymorphic or amorphous form.

A third aspect of the invention provides a compound of the first aspect for use in the treatment of a bacterial infection.

A fourth aspect of the invention provides a compound of the first aspect for use in the inhibition of 4-methylphenol production by bacteria.

A fifth aspect of the invention provides a compound of the first aspect for use in the treatment of Clostridioides difficile infection.

A sixth aspect of the invention provides a pharmaceutical composition comprising a compound of the first aspect, or a pharmaceutically acceptable salt, solvate or prodrug of the second aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

In the pharmaceutical composition may additionally comprise one or more further active agents.

The pharmaceutical composition may be provided as a part of a kit of parts, wherein the kit of parts comprises the pharmaceutical composition of the fourth aspect of the invention and one or more further pharmaceutical compositions, wherein the one or more further pharmaceutical compositions each comprise a pharmaceutically acceptable excipient and one or more further active agents.

A seventh aspect of the invention provides a compound of the first aspect, or a pharmaceutically acceptable salt, solvate or prodrug thereof, or a pharmaceutical composition, for use in medicine, and/or for use in the treatment or prevention of a disease, disorder or condition. Typically, the use comprises the administration of the compound, salt, solvate, prodrug or pharmaceutical composition to a subject. In one embodiment, the use comprises the co-administration of one or more further active agents.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptoms, the amelioration or palliation of the condition/symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition.

An eighth aspect of the invention provides the use of a compound of the first aspect, or a pharmaceutically effective salt, solvate or prodrug of the second aspect, in the manufacture of a medicament for the treatment or prevention of a disease, disorder or condition. Typically, the treatment or prevention comprises the administration of the compound, salt, solvate, prodrug or medicament to a subject. In one embodiment, the treatment or prevention comprises the co-administration of one or more further active agents.

A ninth aspect of the invention provides a method for inhibiting the production of 4-methylphenol by a bacteria using a compound of the first aspect. The method may comprise inoculating a sample comprising the bacteria with the compound.

An tenth aspect of the invention provides a method of treatment or prevention of a disease, disorder or condition, the method comprising the step of administering an effective amount of a compound of the first aspect, a pharmaceutically acceptable salt, solvate or prodrug of the compound, or a composition comprising the compound, to thereby treat or prevent the disease, disorder or condition. In one embodiment, the method further comprises the step of co-administering an effective amount of one or more further active agents. Typically, the administration is to a subject in need thereof.

### Experimental

The suitability of the following compounds, EX1 to EX8, for reducing the proliferation of the bacteria C. difficile, and thus the treatment of C. difficile infections, was investigated.

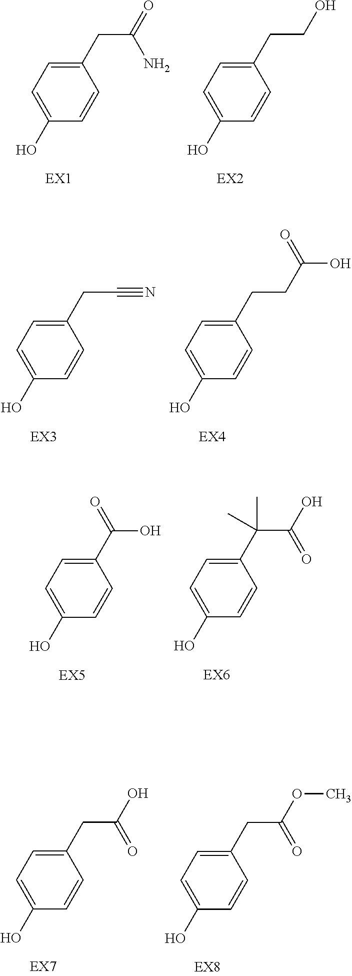

Toxicity to C. difficile

Compounds EX1 to EX5 were separately tested to determine their toxicity to C. difficile and E. coli.

Each compound was dissolved to 6.6 mM in minimal media (MM) with 1% DMSO based on the MM described by Cartman et al. in Appln. Environ. Microbiol, 2010, 76, 1103 and the concentration was matched in a 1:1 ratio of the compound to the substrate p-HPA.

To determine growth rates of C. difficile and E. coli in the presence of compounds EX1 to EX5, three colonies of C. difficile or three colonies of E. coli were used to inoculate a primary culture of 10 mL MM in 50 cm³ vented tissue culture flasks (Thermo Scientific). Following overnight incubation on a shaking platform at 50 rpm, the primary culture was back diluted in MM to OD_{590 nm} 0.5 using a Fisherbrand™ Digital Colorimeter model 4.5 (Fisher Scientific). 1 ml of the OD_{590 nm} 0.5 culture was inoculated into 10 ml of MM in 50 cm³ vented tissue culture flasks with and without 4-hydroxyphenylacetic acid and the relevant compound (EX1 to EX5).

OD_{590 nm} readings were determined every hour for eight hours using a Fisherbrand™ Digital Colorimeter model 4.5 (Fisher Scientific).

Growth curves denoting growth over an 8-hour time period for C. difficile and E. coli in MM with the presence of 4-hydroxyphenylacetic acid and inhibitors EX1 to EX5, each at 6.6 mM. Absorbance was measured at OD_{590 nm}. *p<0.05, **p<0.01, samples represent a minimum of three biological replicates graphed in GraphPad prism (v9). Raw absorbance was normalised using a blank MM control.

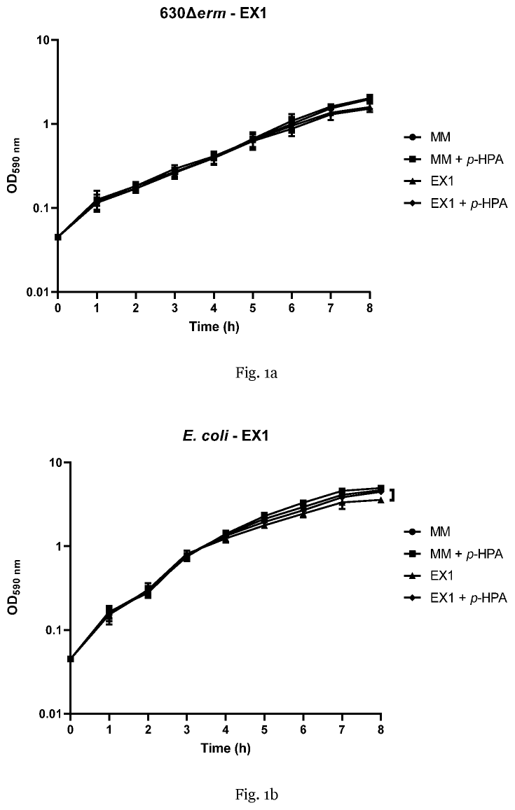

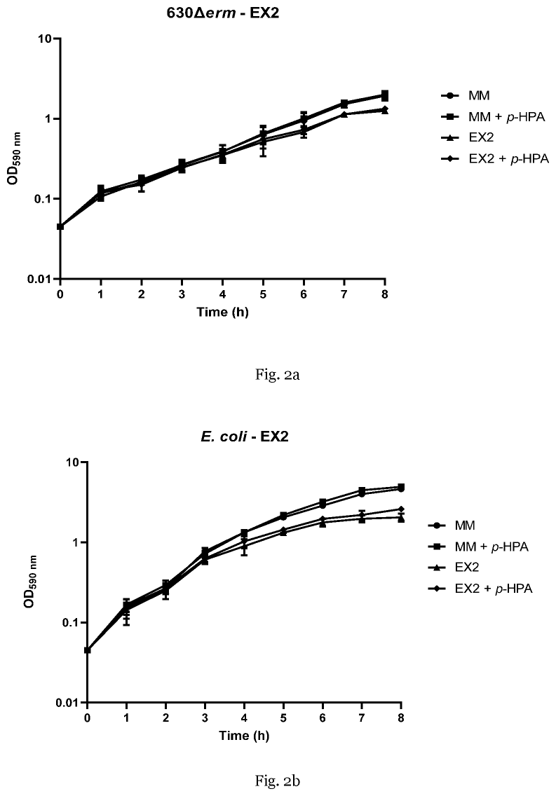

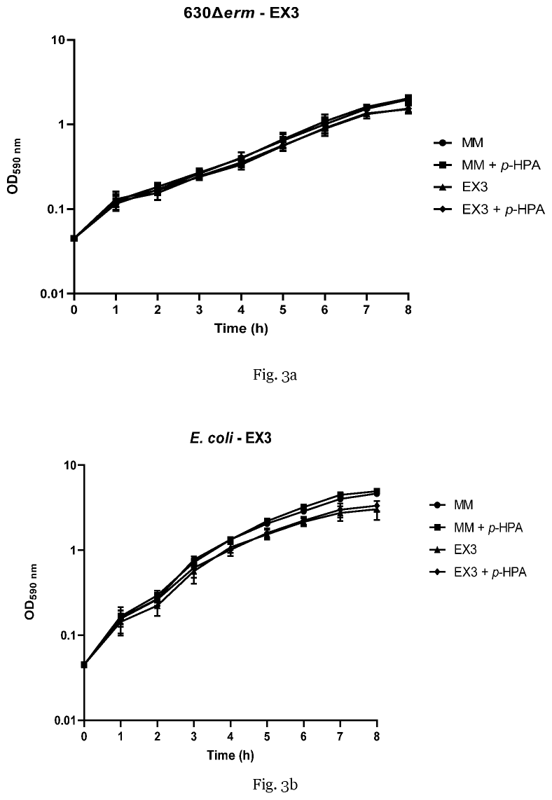

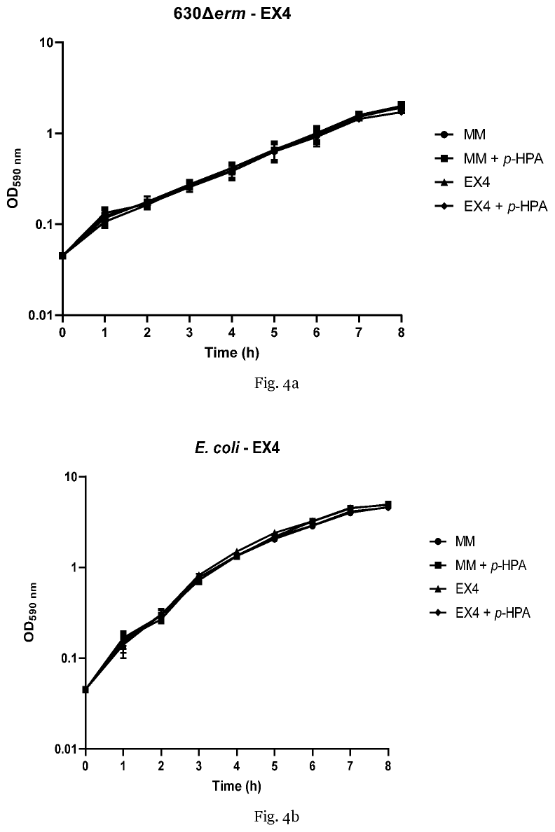

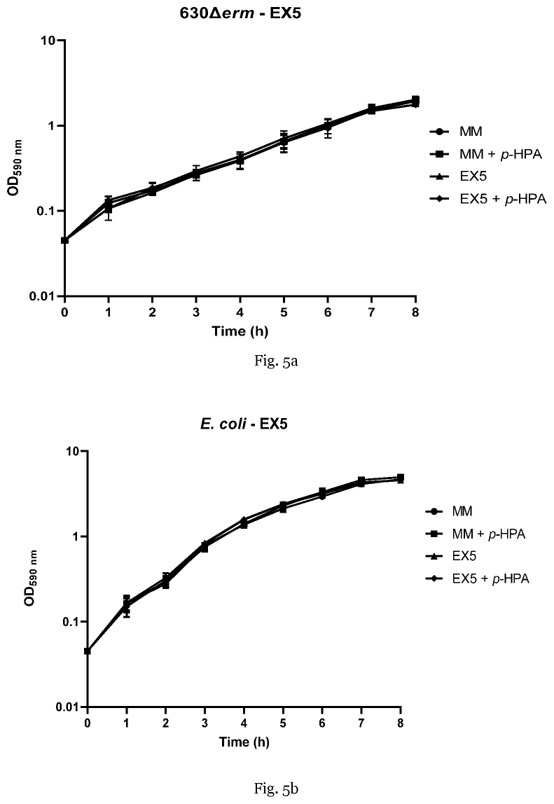

The growth curves are shown in FIGS. 1a to 5b.

The data show that EX1 inhibited the growth of E. coli and had no effect on the growth of C. difficile. EX2 was identified as being toxic to both E. coli and C. difficile. EX3 inhibited the growth of C. difficile when both EX3 and p-HPA were present and inhibited the growth of E. coli. EX4 had no effect on the growth of C. difficile. EX5 had no effect on the growth of either C. difficile or E. coli.

Co-Culture (Mutated C. difficile)

In order to evaluate whether reducing 4-methylphenol production by C. difficile would result in a reduction in the growth of C. difficile, co-cultures comprising C. difficile, a 4-methylphenol deficient mutant of C. difficile and E. coli were developed and used as a surrogate readout of 4-methylphenol production.

Under anaerobic conditions, individual overnight cultures of C. difficile and an intestinal competitor, E. coli, were grown in 10 ml of BHI+0.05% (w/v) L-cysteine supplemented with 100 ng/ml or 250 ng/ml anhydrotetracycline (to induce expression of hpdBCA expressed in trans in the complement strain). Individual monocultures were normalised to a starting optical density (OD₆₀₀) of 0.5, and were inoculated 1/10 into BHIS broth supplemented with 0.05% L-cysteine (w/v) and either 0.1, 0.2 or 0.3% p-HPA (w/v) (6.6 mM, 13.1 mM and 19.7 mM, respectively), these monocultures were grown until C. difficile reached an OD 0.5-0.6 (˜7 hours). The competitor was back diluted to an OD₆₀₀ 0.5 and inoculated 1:10 into the C. difficile culture, to create a competitive co-culture. These co-cultures were grown anaerobically, shaking (50 rpm) for 24 hours and were plated onto both BHIS non-selective plates and BHIS with D-cycloserine (250 mg/L) and cefoxitin (8 mg/L) (CC) plates. CFU counts of both C. difficile and the competitor were determined by serial dilutions plated in triplicate and an average of the three technical replicates was used to determine total CFU. Each experiment was performed in triplicate and linear regression analysis was performed in Stata15 on the log₁₀ of the CFU, statistically significant differences were observed p<0.05.

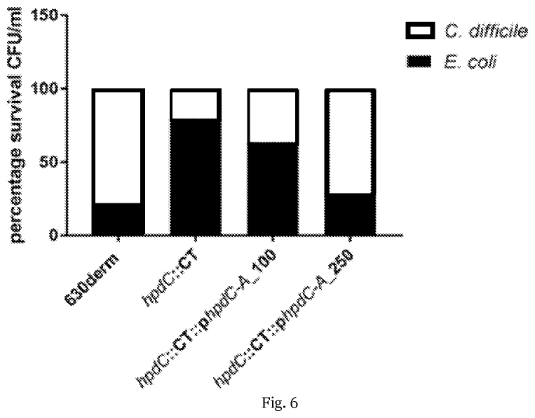

The results are shown in FIG. 6. The data show that E. coli is sensitive to 4-methylphenol and so reduced 4-methylphenol production by C. difficile corresponds to an increase in the growth of E. coli. If C. difficile is mutated to prevent 4-methylphenol production, the viability of C. difficile relative to E. coli is decreased.

### Co-Cultures (Compounds EX1 to EX7)

Compounds EX1 to EX7 were tested to determine their ability to reduce the growth of C. difficile relative to E. coli.

C. difficile 630Δerm and E. coli were grown overnight in MM. C. difficile was back diluted to an OD_{590 nm} of 0.2. 18 μl of the back dilution was added to 1.8 ml of the test conditions: MM, MM+6.6 mM p-HPA, MM+6.6 mM inhibitor, and MM+6.6 mM p-HPA+6.6 mM inhibitor, in a 24 well plate. Each inhibitor was dissolved in DMSO such that the final concentration in the 24 well plate was 1% v/v. C. difficile was grown for 8 hours before E. coli underwent back dilution to an OD_{590 nm} of 0.2 and 18 μl was inoculated into the C. difficile wells to give the co-culture. The co-culture was grown for 14 hours. The proportion of the co-cultures made up by C. difficile and E. coli were determined by colony-forming units per millilitre (CFU/ml) assays with co-cultures plated on to BHIS plates in duplicate with selective media for each species. C. difficile was selected for using cycloserine (250 mg/l) and cefoxitin (8 mg/l) whilst E. coli was selected for using vancomycin (4 mg/l). CFU serial ten-fold dilutions of the co-culture were made in PBS with dilutions to 10⁻⁶. CFUs were counted on the following day. CFU data is presented using CFU percentages which were calculated by dividing the number of CFUs for each species by the total number of CFUs for both E. coli and C. difficile and multiplying by 100. Each experiment was performed in a minimum of three independent replicates. Data was analysed by linear regression carried using Stata17.

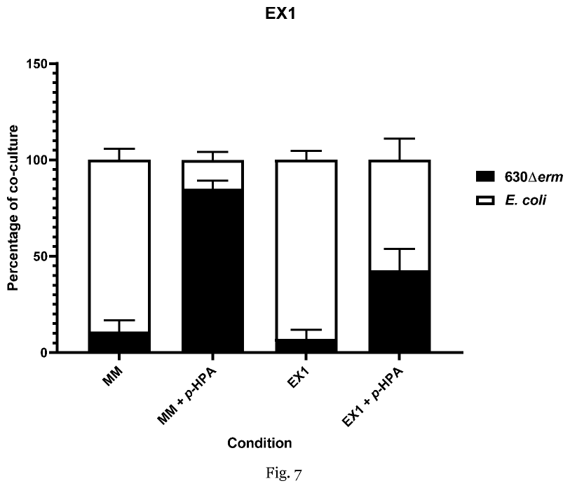

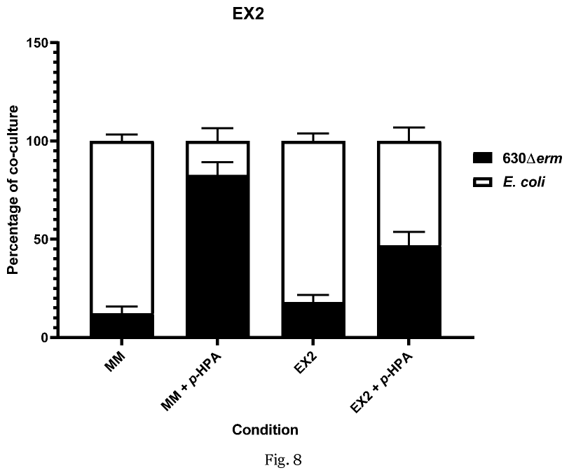

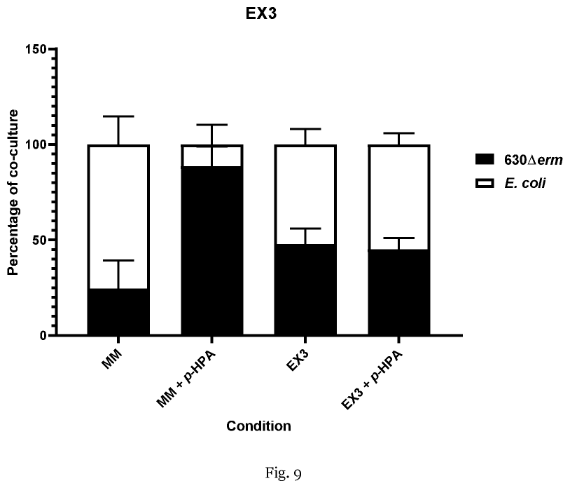

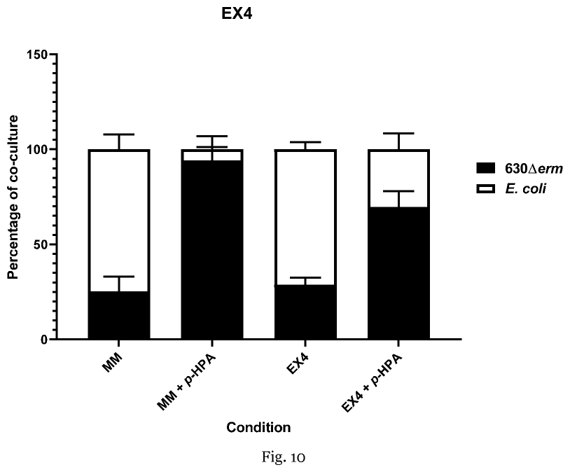

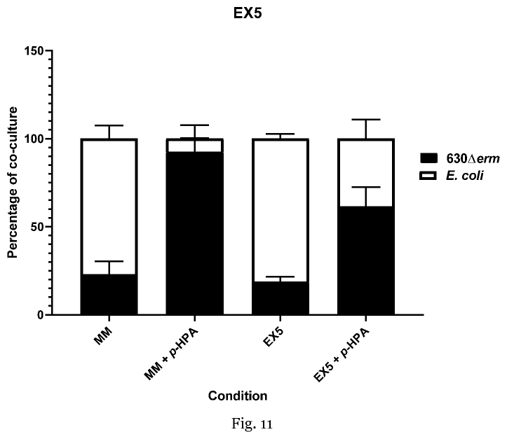

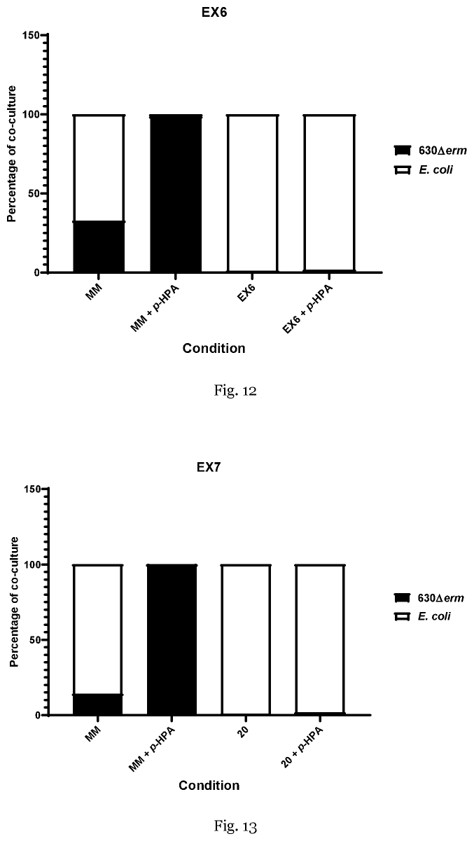

The results are shown in FIGS. 7 to 13.

The data show that each of compound EX1 to EX5 is able to inhibit the growth of C. difficile. Based on this, these compounds would be expected to be effective in maintaining gut microbial diversity and preventing relapse of C. difficile infection. Compounds EX6 and EX7 were also able to inhibit the growth of C. difficile with little or no negative impact on the growth of E. coli. Moreover, all of the compounds had minimal effect on the proportions of the co-cultures when p-HPA was not present. This supports that the effect was as a result of 4-methylphenol production inhibition because 4-methylphenol is only produced in the presence of p-HPA or from tyrosine fermentation to p-HPA.

### HPLC (Monocultures)

C. difficile monocultures were established in the presence of the following compounds EX1, EX3, EX5 and EX8.

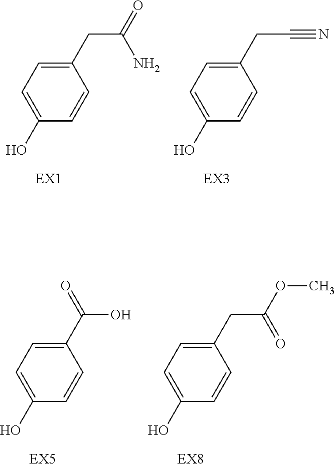

Samples of 630Δerm were collected following growth in minimal media with 1% DMSO v/v for 8 hours with 1.5 mg/ml p-HPA with matched (6.6 mM) concentrations of compounds EX1, EX3, EX5 and EX8. At the time of sample collection, the OD₅₉₀ nm of the culture was measured. Samples were immediately filter sterilised using 0.22 μm filters, then frozen at −80° C. prior to HPLC analysis.

The filter-sterilized samples were transferred to HPLC vials and analysed immediately by injecting onto the HPLC column. Separations were achieved by utilizing an Acclaim 120 (Thermofisher), C₁₈, 5 μm (4.6×150 mm), with the mobile phase consisting of ammonium formate (10 mM, pH 2.7) and menthol (v/v; 50:50) at a flow rate of 1400 μl/min. p-HPA and p-cresol were detected by the diode array detector (PDA; DAD 3000) set at 280 nm. Peak identity was confirmed by measuring the retention time of commercially available p-HPA and p-cresol, and determination of absorbance spectra was performed using the DAD. A calibration curve of each compound was generated by Chromeleon (Dionex software) using known amounts of the reference standards (0-5 mg/ml) dissolved in media and injected onto the column, and the amount of p-HPA and p-cresol in the samples was determined. Samples from three independent biological replicates were analysed compared to media controls and standard curves. The limit of detection for p-HPA and p-cresol are 0.001 and 0.0005 mg/ml, respectively. The p-cresol concentration was normalised to growth by using the OD_{590 nm} measured at the time of sample collection.

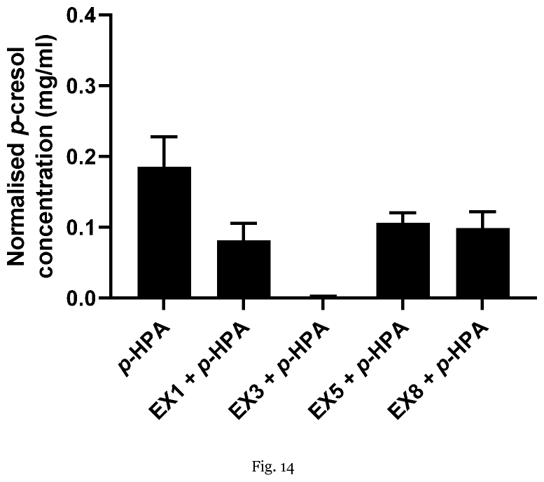

The results are shown in FIG. 14.

The HPLC data demonstrate that the production of 4-methylphenol by C. difficile is reduced in the presence of compounds EX1, EX3, EX5 and EX8. The reduced 4-methylphenol production is indicative that these compounds are capable of inhibiting the production of 4-methylphenol by C. difficile. By inhibiting the production of 4-methylphenol, other gut bacteria, such as E. coli, would be expected to be able to effectively compete against C. difficile.

### HPLC (Co-Cultures)

A C. difficile 630Δerm and E. coli co-culture was established (as per the methods previously described) in the presence of compound EX3.

Following co-culture, CFU assays were undertaken to determine relative survival of C. diff and E. coli. The remaining sample was filter sterilised (0.22 μm filters), then frozen at −80° C. prior to the HPLC analysis. HPLC analysis was carried out as described above (HPLC monocultures) with the exception of there being no normalisation of the 4-methylphenol concentration to OD_{590 nm}.

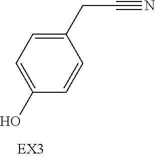

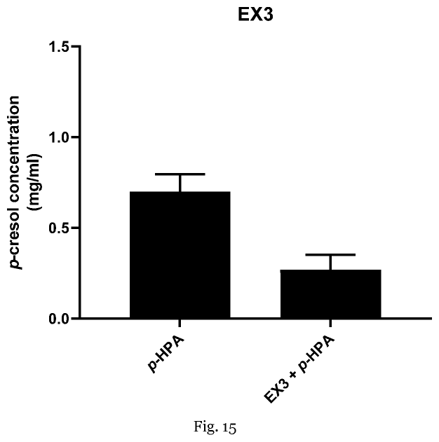

The results are shown in FIG. 15. The HPLC co-culture data demonstrate that the production of 4-methylphenol by C. difficile is reduced in the presence of compound EX3 and E. coli.

1. A compound of Formula (I): 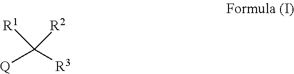
or a pharmaceutically acceptable salt, solvate or prodrug thereof, wherein:
Q is a substituted or unsubstituted cyclic group;
each of R¹ and R² together represent ═O or is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}; and
R³ is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, R^{α} C(O)OR^{α}, R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α};
wherein each R^{α} is independently selected from a methylene C₁-C₂₀ alkyl, C₂-C₂₀ alkenyl, C₂-C₂₀ alkynyl or C₃-C₂₀ cyclic group;
wherein any —R^{α} may optionally be substituted with one or more methylene, C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups and wherein each n is independently an integer from 1 to 20.

2. The compound of claim 1, wherein Q is a substituted or unsubstituted 3- to 10-membered cyclic group.

3. The compound of claim 1, wherein Q is a substituted or unsubstituted 5-membered or 6-membered cyclic group, optionally wherein the cyclic group is a heterocyclic group.

4. The compound of claim 1, wherein Q is a substituted or unsubstituted aryl, cyclohexyl, pyridyl or piperidinyl group.

5. The compound of claim 1, wherein each of R¹ and R² together represent ═O or R¹ is independently selected
—H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α} and R² is independently selected from —H, —R^{α}, —Cl, —Br, —I, —F, —OH, —R^{α}OH, —OC(O)R^{α}, —OR^{α}, —OCH₂NHR^{α}, —OCHR^{α}NR^{α}₂, —OCH₂R^{α}NHR^{α}, —NO₂, —NH₂, —N₃, —SH, —SO₂H, —SO₂NH₂, —CO₂H, —R^{α}CN, —CN, —C(O)NH₂, —C(O)NHR^{α}, —C(O)NR^{α}₂, —C(O)OR^{α}, —R^{α} C(O)OR^{α}, —R^{α} C(O)OH, —CH₃, —CₙH₂ₙ₋₂(O)NH₂, —CₙH₂ₙ₋₂(O)NHR^{α}, or —CₙH₂ₙ₋₂(O)NR^{α}R^{α}, wherein each R^{α} is independently selected from a methylene, C₁-C₂₀ alkyl, C₂-C₂₀ alkenyl, C₂-C₂₀ alkynyl or C₃-C₂₀ cyclic group, wherein any —R^{α} may optionally be substituted with one or more methylene, C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups and wherein each n is independently an integer from 1 to 20.

6. The compound of claim 1, wherein R³ is —R^{α}OH, —OH, —CO₂R^{α}, —R^{α} CO₂R^{α}, —R^{α} CO₂H, —CH₃, —CN, —R^{α}CN,
—CₙH₂ₙ₋₂(O)NH₂, —CₙH₂ₙ₋₂(O)NHR^{α} or —CₙH₂ₙ₋₂ON(R^{α})R^{α}.

7. The compound of claim 1, wherein n is an integer from 1 to 5.

8. The compound of claim 1, wherein R³ is —OH, —COOH, —C(O)OCH₃, —C(O)OCH₂CH₃, —CH₂C(O)OCH₃,
—CH₂C(O)OCH₂CH₃, —CH₂C(O)OH, —CH₂CH₂C(O)OH, —CH₂CH₂C(O)CH₃, —CH₂CH₂C(O)OCH₂CH₃, —CH₃, —CN, —CH₂CN, —C₂H₄CN, —C(O)NH₂, —C(O)NCH₃, —C(O)N(H)CH₂CH₃, —CH₂(O)NH₂, —CH₂(O)NHCH₃ or —CH₂(O)N(CH₃)CH₃.

9. The compound of claim 1, wherein the compound is selected from the group consisting of:

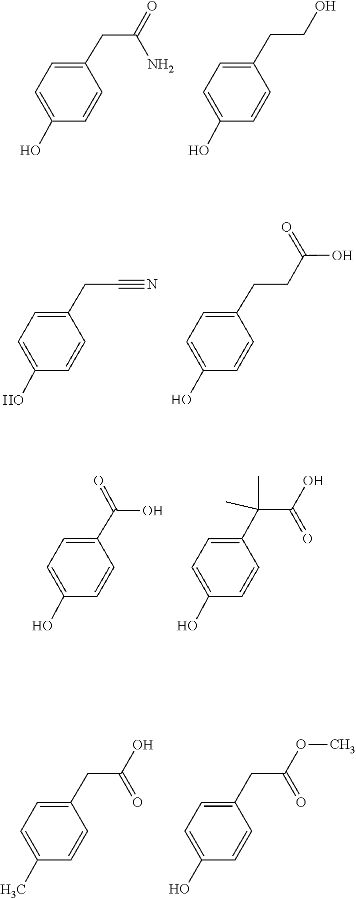

10. The compound of claim 1, wherein the medicament is for use in the treatment of a bacterial infection.

11. The compound of claim 1, wherein the medicament is for the inhibition of 4-methylphenol production by bacteria.

12. The compound of claim 1, wherein the medicament is for use in the treatment of Clostridioides difficile (C. difficile) infection.

13. A pharmaceutically acceptable salt, solvate or prodrug of a compound comprising the compound of claim 1.

14. A pharmaceutical composition comprising the compound as claimed in claim 1, or a pharmaceutically acceptable salt, solvate or prodrug as claimed in claim 13, and a pharmaceutically acceptable excipient.

15. A method of inhibiting the production of 4-methylphenol by a bacteria, the method comprising administering of the compound as claimed in claim 1, a pharmaceutically acceptable salt, solvate or prodrug as claimed in claim 13, or a composition as claimed in claim 14 to a subject in need thereof.

16. A method of treatment or prevention of a disease, disorder or condition, the method comprising administering an effective amount of a compound as claimed in claim 1, a pharmaceutically acceptable salt, solvate or prodrug as claimed in claim 13, or a pharmaceutical composition as claimed in claim 14 to a subject in need thereof.
